(19) United States
(12) Patent Application Publication
(10) Pub. No.: US 2019/0298192 A1
(43) Pub. Date: Oct. 3, 2019

(54) LIVING BODY MEASUREMENT APPARATUS AND COMPUTER-READABLE STORAGE MEDIUM

(71) Applicant: CANON KABUSHIKI KAISHA, Tokyo (JP)

(72) Inventors: Hitoshi Furukawa, Suntou-gun (JP); Nobutoshi Sugai, Numazu-shi (JP)

(21) Appl. No.: 16/364,261

(22) Filed: Mar. 26, 2019

(30) Foreign Application Priority Data
Mar. 27, 2018 (JP) 2018-060732
Mar. 27, 2018 (JP) 2018-060735

(51) Int. Cl. A61B 5/021; A61B 5/00

(52) U.S. Cl. CPC A61B 5/02125; A61B 5/0075

(57) ABSTRACT

A measurement apparatus includes: a spectral sensor including a plurality of pixels; a unit configured to generate a biological signal from a light reception result of a predetermined pixel; a unit configured to detect a feature amount of the biological signal; a unit configured to set a measurement condition to the spectral sensor; a unit configured to determine whether or not the measurement condition needs to be changed based on the biological signal; and a unit configured to determine, if the measurement condition needs to be changed, in a first cycle of the biological signal, whether or not the measurement condition can be changed in a period from a timing at which the detection unit has detected the feature amount in the first cycle until an ending time of the first cycle.

### BACKGROUND OF THE INVENTION

### Field of the Invention

The present invention relates to a living body measurement apparatus and a computer-readable storage medium.

### Description of the Related Art

A measurement apparatus is known that illuminates a portion of a living body with light and detects biological information by detecting an amount of light reflected from the living body or an amount of light that has passed through the portion of the living body. The biological information refers to various types of physiological/anatomical information that can be obtained from a living body such as a pulse rate and a degree of blood vessel stiffness, for example. For example, the pulse rate can be detected based on a pulse wave signal that indicates variation of an amount of reflected or transmitted light that is caused by blood movement inside a blood vessel. Also, the degree of blood vessel stiffness can be detected based on a feature point of an acceleration pulse wave signal (hereinafter, simply referred to as an acceleration signal) obtained by differentiating the pulse wave signal twice, for example.

Japanese Patent Laid-Open No. 2004-000467 discloses a pulse wave measuring apparatus, which is an example of the biological information measurement apparatus. According to Japanese Patent Laid-Open No. 2004-000467, the pulse wave measuring apparatus detects the pulse wave by illuminating a fingertip portion with a luminous flux and detecting a temporal change in the amount of reflected light. When detecting biological information, if the measurement state of a living body changes after the measurement has been started, the measurement accuracy may decrease. Therefore, Japanese Patent Laid-Open No. 2010-004972 discloses a configuration in which, if the measurement state of a living body has changed during measurement, the measurement condition such as a light emission time period and a light emission interval of the light emission pulse is switched. Also, Japanese Patent Laid-Open No. 2017-108905 discloses a configuration in which power consumption of the measurement apparatus is reduced by switching the operation mode of a light receiving unit based on a timing of a feature point of the detection signal detected by the light receiving unit. Japanese Patent Laid-Open No. 2013-150772 discloses a configuration in which a plurality of pieces of biological information are obtained by using a light receiving unit including a plurality of light receiving elements. Furthermore, Japanese Patent Laid-Open No. 2015-000127 discloses a configuration in which carboxyhemoglobin concentration is measured.

If the measurement condition is switched while biological information is being detected, the detection signal detected by the light receiving unit changes, and it is difficult to detect the biological information from the detection signal due to the change in the detection signal. On the other hand, if the measurement is suspended in order to switch the measurement condition, consecutive detection of the biological information is interrupted, and the detection time of the biological information increases. Although Japanese Patent Laid-Open No. 2010-004972 discloses a configuration in which the measurement condition is switched, the configuration that makes it possible to avoid the influence of the variation in the detection signal caused by the switching is not disclosed. Japanese Patent Laid-Open No. 2017-108905 is aimed at reducing power consumption, and does not disclose a configuration in which the measurement condition is switched according to the change in the measurement state of a living body, and the influence of the variation in the detection signal caused by the switching can be avoided.

Also, when a plurality of pieces of biological information are detected, an appropriate measurement condition may differ according the biological information to be detected. Therefore, when a plurality of pieces of biological information are detected in parallel using a common light source and a common light reception sensor, the measurement condition needs to be switched during measurement. Although Japanese Patent Laid-Open No. 2013-150772 discloses a configuration in which a plurality of pieces of biological information are detected, a configuration in which the measurement condition is switched in order to detect the plurality of pieces of biological information is not disclosed.

### SUMMARY OF THE INVENTION

According to an aspect of the present invention, a measurement apparatus includes: a spectral sensor that includes a light source configured to emit light toward a measurement position, a spectral unit configured to disperse reflected light from a living body at the measurement position or transmitted light that has passed through a living body at the measurement position according to a wavelength, and a light receiving unit including a plurality of pixels, wherein each pixel of the plurality of pixels is configured to receive light having a predetermined wavelength that has been dispersed by the spectral unit; a generation unit configured to generate a biological signal from a light reception result of a predetermined pixel of the light receiving unit; a first determination unit configured to determine a cycle of the biological signal; a detection unit configured to detect a feature amount of the biological signal; a setting unit configured to set a measurement condition of the living body to the spectral sensor; a second determination unit configured to determine whether or not the measurement condition needs to be changed based on the biological signal; and a third determination unit configured to determine, if the second determination unit has determined that the measurement condition needs to be changed, in a first cycle of the biological signal, whether or not the measurement condition can be changed in a period from a timing at which the detection unit has detected the feature amount in the first cycle until an ending time of the first cycle.

Further features of the present invention will become apparent from the following description of exemplary embodiments with reference to the attached drawings.

### BRIEF DESCRIPTION OF THE DRAWINGS

FIG. 1 is a functional block diagram of a measurement apparatus according to one embodiment.

FIG. 2 is a diagram illustrating a configuration of the measurement apparatus according to one embodiment.

FIG. 3A is a diagram illustrating a spectrum of a white light source according to one embodiment.

FIG. 3B is a diagram illustrating a configuration of a line sensor according to one embodiment.

FIG. 4 is a flowchart of processing for detecting biological information according to one embodiment.

FIG. 5A is a diagram illustrating a biological signal according to one embodiment.

FIG. 5B is a diagram illustrating an acceleration signal according to one embodiment.

FIG. 5C is a diagram illustrating feature points according to one embodiment.

FIG. 6 is a flowchart of processing for changing a measurement condition according to one embodiment.

FIGS. 7A to 7C are diagrams illustrating processing for changing the measurement condition according to one embodiment.

FIG. 8 is a flowchart of processing for changing the measurement condition according to one embodiment.

FIGS. 9A to 9C are diagrams illustrating processing for changing the measurement condition according to one embodiment.

FIG. 10 is a functional block diagram of a measurement apparatus according to one embodiment.

FIG. 11A is a diagram illustrating a spectrum of a white light source according to one embodiment.

FIG. 11B is a diagram illustrating a configuration of a line sensor according to one embodiment.

FIG. 12 is a flowchart of processing for detecting biological information according to one embodiment.

FIG. 13 is a diagram illustrating detection processing according to one embodiment.

FIG. 14 is a flowchart of processing for detecting biological information according to one embodiment.

FIGS. 15A and 15B are diagrams illustrating an external appearance of a measurement apparatus according to one embodiment.

### DESCRIPTION OF THE EMBODIMENTS

Hereinafter, illustrative embodiments of the present invention will be described with reference to the drawings. Note that the following embodiments are illustrative and do not limit the present invention to the contents of the embodiments. Also, in the following diagrams, constituent elements that are not required for describing the embodiments are omitted.

### First Embodiment

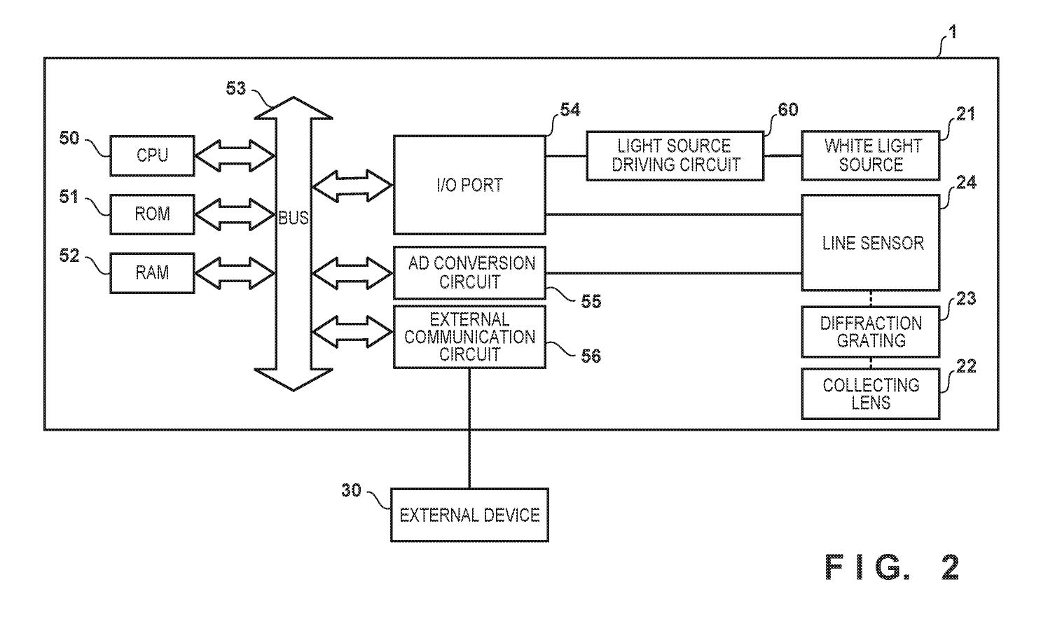

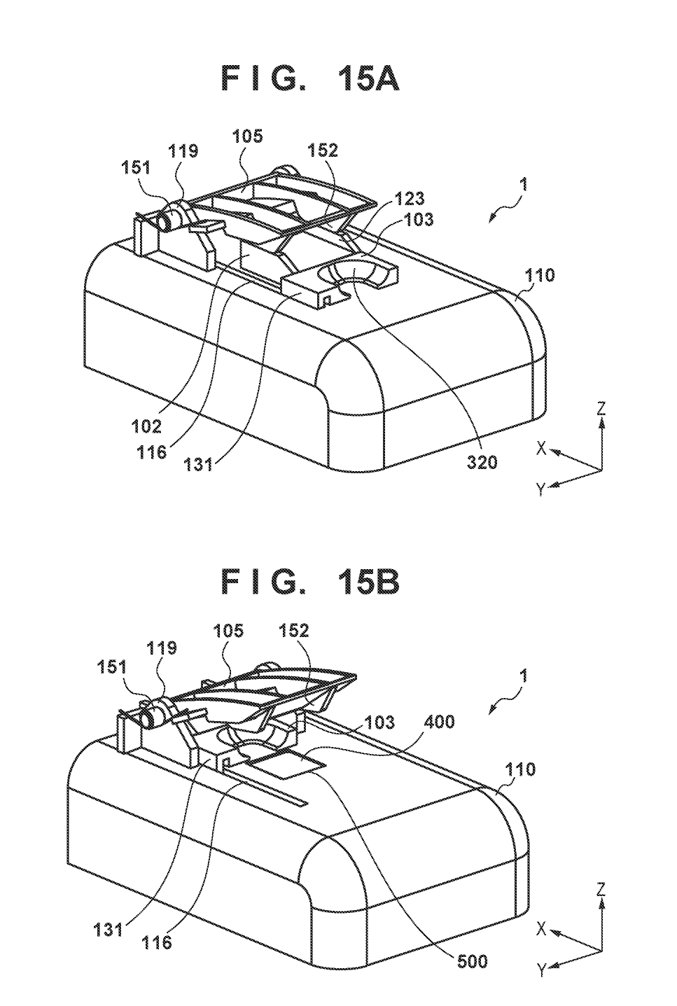

FIGS. 15A and 15B are perspective views of a measurement apparatus 1 according to the present embodiment. Note that FIG. 15A shows a state in which a shutter member 102 covers an aperture portion 500, and FIG. 15B shows a state in which the shutter member 102 has been moved to a retreat position, and the aperture portion 500 is exposed. Note that the aperture portion 500 is covered by a transparent cover 400 for preventing foreign matters from falling inside a housing 110. The housing 110 is provided with a groove-like guide rail 116 that runs along an X direction in the diagram. Also, a guide portion 131 of a guide member 103 is fitted into this guide rail 116. With this, the guide member 103 and the shutter member 102 can move in the X direction in a range in which the guide rail 116 is provided. Note that a spring is attached, inside the housing 110, to the guide portion 131 of the guide member 103. This spring force keeps the guide member 103 at the position shown in FIG. 15A when an external force is not applied to the guide member 103. When a user measures biological information, the user pushes a finger receiving portion 320 of the guide member 103 in the X direction with a finger, and slides the guide member 103 and the shutter member 102 in the X direction. The measurement apparatus 1 is configured such that, when the guide member 103 and the shutter member 102 are pushed by a finger until the position limited by the guide rail 116, a tip portion of the finger covers the aperture portion 500. In this state, a white light source 21 (FIG. 2) illuminates the finger with light from inside the housing 110 via the aperture portion 500, and a line sensor 24 (FIG. 2) inside the housing 110 receives the reflected light.

Also, the housing 110 is provided with a press member 105 and a pressing spring 151 that are rotatably held by a bearing portion 119. The pressing spring 151 applies a force toward the aperture portion 500 to the press member 105. Also, the press member 105 is provided with a pressing rib 152, and the shutter member 102 is provided with a pressed rib 123. In the state shown in FIG. 15A, the pressing rib 152 comes into contact with the pressed rib 123, and the shutter member 102 is biased toward an upper surface of the housing 110. Also, a white reference plate is provided on a face of the shutter member 102 on the aperture portion 500 side. This white reference plate is used to calibrate the white light source 21, the line sensor 24, and the like that are inside the housing 110. The shutter member 102 prevents light from leaking from the housing 110 to the outside thereof via the aperture portion 500 when calibration is performed. Also, the press member 105 has a role of stabilizing a fingertip, which is the measurement target, at a position of the aperture portion 500 during measurement.

FIG. 2 is a diagram illustrating a configuration of hardware arranged inside the housing 110 of the measurement apparatus 1. A CPU 50 is a control unit that performs overall control of the measurement apparatus 1. The CPU 50 executes later-described various types of control based on a program stored in a ROM 51. Note that the CPU 50 stores data that is used when the various types of control are executed and data that needs to be temporarily stored to a RAM 52. The CPU 50 can communicate with the ROM 51, the RAM 52, an I/O port 54, an AD conversion circuit 55, and an external communication circuit 56 via a bus 53. A light source driving circuit 60 controls light emission of the white light source 21. Also, the CPU 50 can control the light emission intensity of the white light source 21 by controlling the light source driving circuit 60 via the I/O port 54. Moreover, the CPU 50 can set a charge accumulation time period of the line sensor 24 via the I/O port 54. As will be described later, the line sensor 24 receives reflected light of light emitted from the white light source 21 via a collecting lens 22 and a diffraction grating 23, and outputs a voltage corresponding to the received light amount to the AD conversion circuit 55. Then, the CPU 50 obtains the voltage corresponding to the received light amount that is output from the line sensor 24 via the AD conversion circuit 55. Moreover, the CPU 50 is configured to be able to communicate with an external device 30 via an external communication circuit 56. Note that the collecting lens 22, the diffraction grating 23, and the line sensor 24 constitute a spectral colorimeter (spectral sensor). Alternatively, the white light source 21, the collecting lens 22, the diffraction grating 23, and the line sensor 24 constitute a spectral sensor.

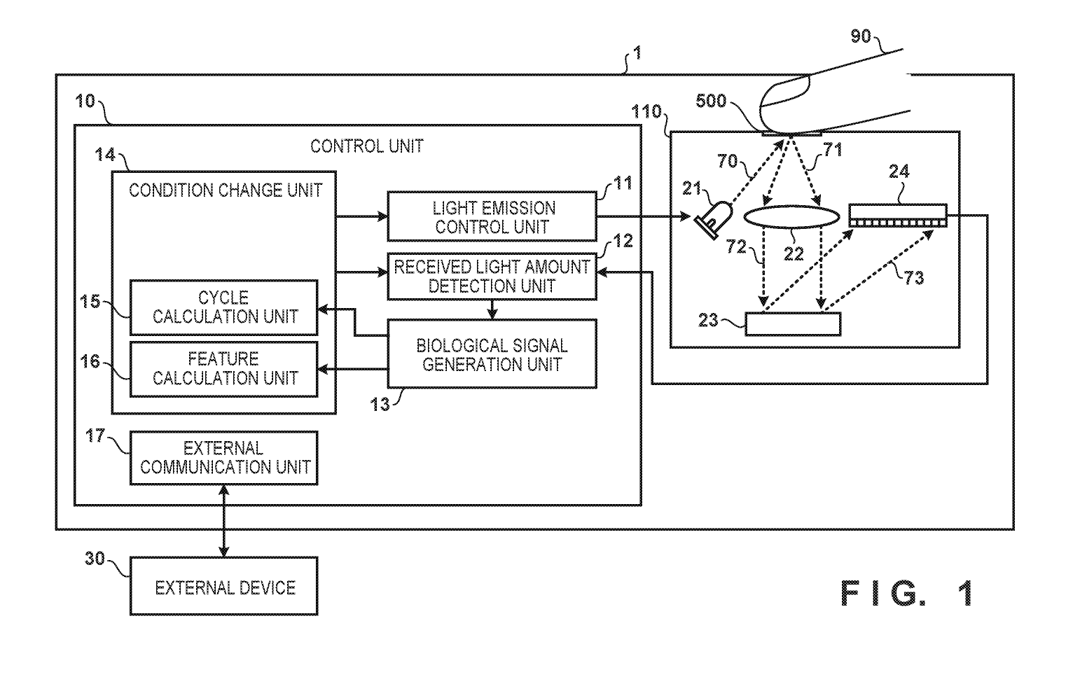

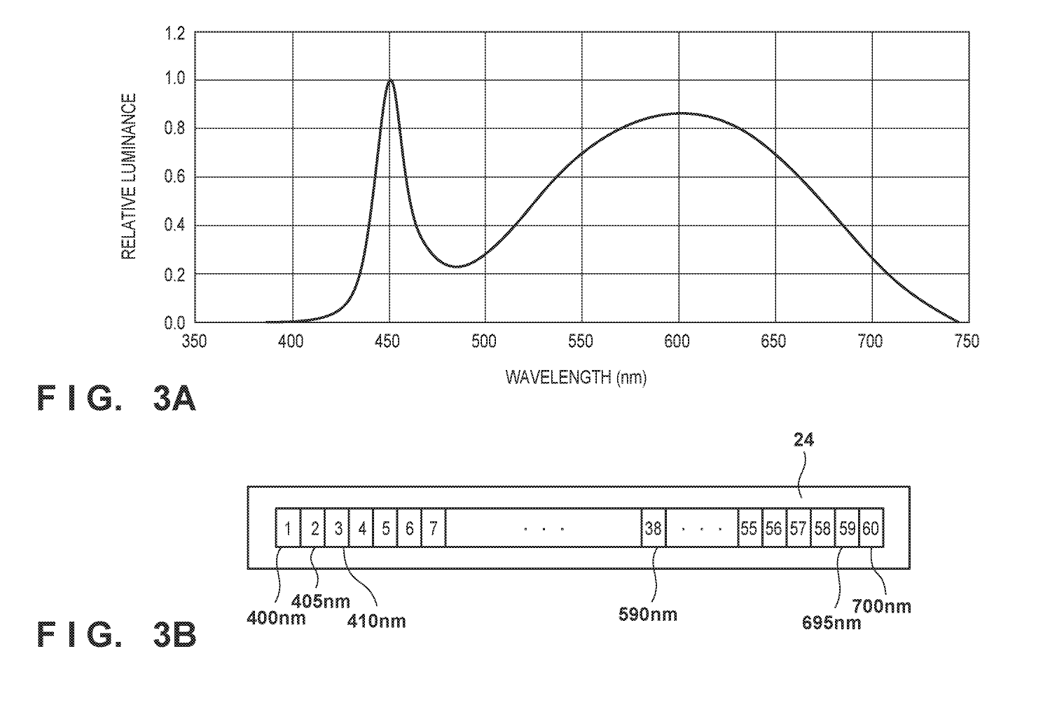

FIG. 1 is a block diagram illustrating operations of the measurement apparatus 1 in the present embodiment. The CPU 50 functions as a control unit 10 in FIG. 1, by executing a program stored in the ROM 51, in cooperation with the I/O port 54, the light source driving circuit 60, the AD conversion circuit 55, the external communication circuit 56, the ROM 51, and the RAM 52. A light emission control unit 11 corresponds to the CPU 50 and the light source driving circuit 60, adjusts the light emission intensity of the white light source 21, and controls the light emission of the white light source 21. The white light source 21 emits light having wavelength distribution that extends to the entirety of visible light. Tungsten light, a white LED, RGB (red, green, blue) three-color LEDs, or the like can be used as the white light source 21, for example. In the present embodiment, the white light source 21 is a white LED in which an LED element that emits blue light is packaged by a resin that includes a yellow fluorescent material. FIG. 3A shows relative intensity (luminance) of the white light source 21 used in the present embodiment for each wavelength. The peak at a wavelength of around 450 nm is a light emission spectrum of the blue LED, and the peak of around 600 nm is a spectrum of the yellow fluorescent material. This spectrum results from light that is emitted from a fluorescent material due to fluorescence upon receiving light from the LED element.

As shown in FIG. 1, light 70 emitted from the white light source 21 passes through the aperture portion 500 of the housing 110 at an angle of about 45 degrees relative to its normal direction and illuminates a fingertip, which is the measurement target 90, at a measurement position. Then, scattered light 71, which depends on the optical absorption property of the measurement target 90, is generated from the illumination light. A portion of the scattered light 71 is converted to parallel light 72 by the collecting lens 22, and the parallel light 72 is incident on the diffraction grating 23 at an incidence angle of 90 degrees. The diffraction grating 23 disperses the incident light according to the wavelength. The dispersed light 73 that has been dispersed is incident on pixels of the line sensor 24. Each pixel of the line sensor 24 outputs a voltage corresponding to the received light amount of the dispersed light 73 that has been incident thereon to a received light amount detection unit 12. FIG. 3B is a schematic diagram of the line sensor 24. The line sensor 24 according to the present embodiment includes 60 pixels needed to detect visible light in a wavelength range from about 400 nm to about 700 nm in units of 5 nm. In the present embodiment, the measurement apparatus 1 is calibrated and assembled such that a first pixel of the line sensor 24 detects light including light having wavelength of about 400 nm and a 60^{th} pixel detects light including light having wavelength of about 700 nm. The received light amount detection unit 12 corresponds to the CPU 50, the AD conversion circuit 55, and the I/O port 54. In actuality, the AD conversion circuit 55 converts the voltage of each pixel that is output from the line sensor 24 to a 12-bit digital value, for example, and the CPU 50 obtains digital values indicating the received light amount of the respective pixels from the AD conversion circuit 55. The line sensor 24 of the present embodiment is a charge accumulation type, and outputs a voltage signal, for each pixel, according to the light amount of the dispersed light that has been incident on the pixel during a predetermined accumulation time period. The accumulation time period of the line sensor 24 is set to the line sensor 24 by the received light amount detection unit 12, specifically by the CPU 50, via the I/O port 54.

The biological signal generation unit 13 generates a biological signal based on a light reception result of a predetermined pixel, that is, a value indicating a received light amount. In the present embodiment, a fingertip of a living body is the measurement target 90, and the biological signal is generated based on the received light amount of a 38^{th} pixel that detects light including light having a wavelength of about 590 nm. This biological signal is also referenced as a fingertip plethysmogram signal. The wavelength of about 590 nm that is used to generate the biological signal is a wavelength at which an amount of light that is absorbed by hemoglobin in a blood is relatively large.

An external communication unit 17 corresponds to the external communication circuit 56, and communicates with the external device 30. The external device 30 instructs the measurement apparatus 1 to start and end measurement. Also, the measurement apparatus 1 transmits a biological signal, a signal obtained by differentiating the biological signal a plurality of times, a cycle C of the biological signal, feature points of the biological signal, and the like to the external device 30. Note that the external device 30 can calculate the pulse rate from the cycle C of the biological signal. Moreover, the external device 30 can determine the degree of blood vessel stiffness based on the feature points and the values thereof. The external device 30 is a personal computer or a tablet terminal, for example. Note that the communication with the external device 30 may be wired communication or wireless communication. A condition change unit 14 will be described later.

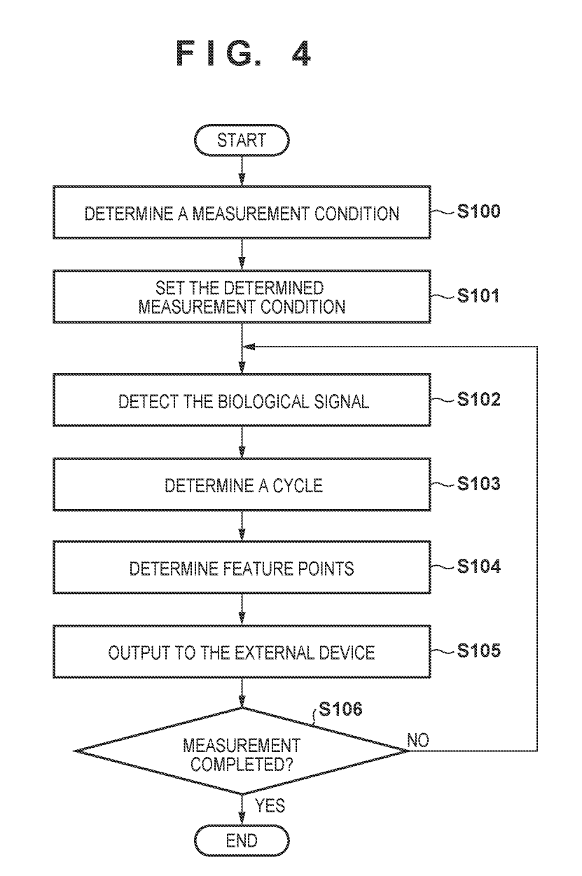

FIG. 4 is a flowchart of processing for detecting the biological information. The measurement apparatus 1, upon receiving an instruction to start measurement from the external device 30, determines a measurement condition in step S100. Note that the measurement condition is a light emission intensity of the white light source 21 and a charge accumulation time period (light receiving time period) of the line sensor 24, for example. For example, the light emission control unit 11 causes the white light source 21 to emit light at a predetermined light emission intensity, and the received light amount detection unit 12 detects the received light amount of the 38^{th} pixel that is used to generate the biological signal during a predetermined period. Also, the control unit 10 determines the light emission intensity of the white light source 21 and/or the charge accumulation time period of the line sensor 24 such that the maximum value of the received light amount detected during the predetermined period is close to the maximum value of the voltage range that can be detected by the AD conversion circuit 55, and sets these values as the measurement condition. Note that the predetermined period is set to be one cycle or more of a fingertip plethysmogram signal, which is the biological signal to be generated, and may be two seconds, for example. Note that, the light emission intensity of the white light source 21 that is used in order to determine the measurement condition in step S100 is determined in advance. The control unit 10 obtains the light emission intensity of the white light source 21 to be used and/or the charge accumulation time period of the line sensor 24 by calculation based on this light emission intensity and the maximum value of the received light amount of the 38^{th} pixel. Also, the configuration may be such that the light emission intensity of the white light source 21 and/or the charge accumulation time period of the line sensor 24 at which the maximum value of the received light amount is appropriate are obtained while changing the light emission intensity of the white light source 21 and/or the charge accumulation time period of the line sensor 24 for each predetermined period. The control unit 10 sets the determined measurement condition to the light emission control unit 11 and the received light amount detection unit 12 in step S101. With this, the light emission control unit 11 causes the white light source 21 to emit light at the light emission intensity that has been determined in step S100. Also, the received light amount detection unit 12 sets the charge accumulation time period that has been determined in step S100 to the line sensor 24.

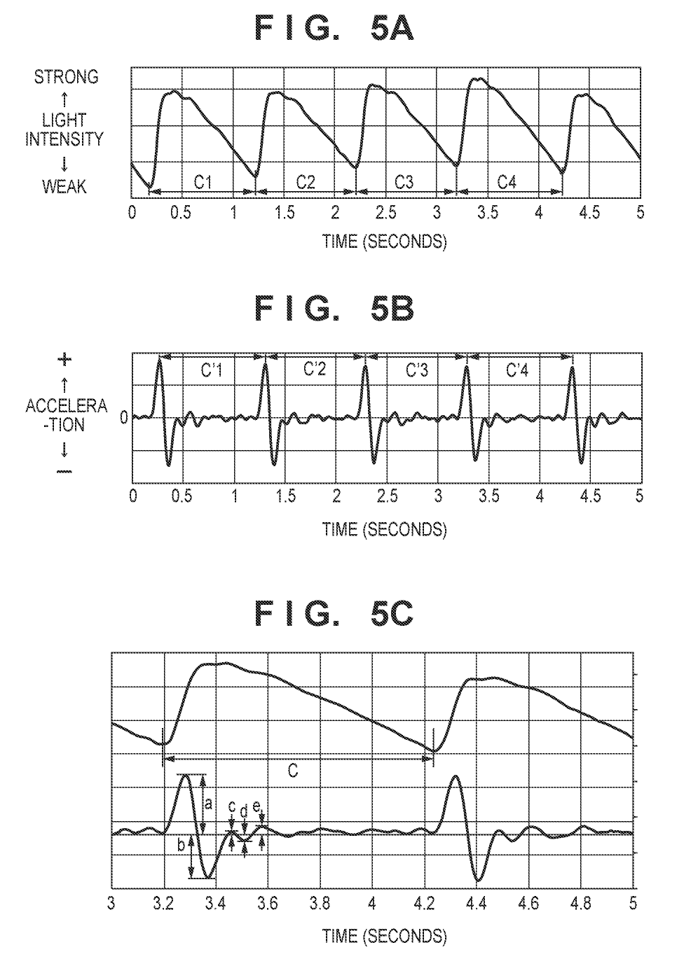

The biological signal generation unit 13 generates a biological signal based on the received light amounts of the 38^{th} pixel at respective timings, and outputs the biological signal to the cycle calculation unit 15 and the feature calculation unit 16. Note that the biological signal generation unit 13 can generate the biological signal by outputting the received light amounts of the 38^{th} pixel at the respective timings as is in time series. Also, the biological signal generation unit 13 can generate the biological signal by sectioning the received light amounts of the 38^{th} pixel at the respective timings at every predetermined number of the received light amounts, obtaining an average value for the predetermined number of the received light amounts, and outputting the average values in the respective sections in time series. Also, the biological signal generation unit 13 can generate the biological signal by obtaining moving averages of received light amounts of the 38^{th} pixel at a predetermined number of timings. Also, the biological signal can be obtained by performing filter processing on signals indicating received light amounts at respective timings or average values thereof in time series. The cycle calculation unit 15 and the feature calculation unit 16 detects the biological signal in step S102. Then, the cycle calculation unit 15 determines the cycle C of the biological signal based on extreme values of the biological signal in step S103. The cycle calculation unit 15 determines the cycle C of the biological signal based on time intervals of local minimums of the biological signal, for example. FIG. 5A shows a detection example of the biological signal and the cycle C (four cycles of C1 to C4) of the biological signal. Also, the cycle calculation unit 15 can also determine the cycle C of the biological signal based on time intervals of local maximums of an acceleration signal obtained by differentiating the biological signal twice. FIG. 5B shows a detection example of the acceleration signal and the cycle C (four cycles of C′1 to C′4) of the biological signal that has been determined based on time intervals of local maximums of the acceleration signal.

Also, the feature calculation unit 16 calculates feature points of the biological signal and values thereof in step S104. In the present embodiment, the feature points are first five local maximums and minimums of the acceleration signal obtained by differentiating the biological signal twice, the start timing of the cycle C of the biological signal being the origin. Note that the start timing of the cycle C of the biological signal is the timing of the local minimum of the biological signal. FIG. 5C shows an example of five feature points a, b, c, d, and e. Note that the feature calculation unit 16 can also determine local maximums and minimums of a signal obtained by differentiating the biological signal four times as the feature points. More generally, the feature calculation unit 16 can determine information regarding change points in a differential signal obtained by differentiating the biological signal one or more times as the feature amounts. Also, the number of feature points is not limited to five, and may be another number. The external communication unit 17 outputs the biological signal, the acceleration signal, the cycle C of the biological signal, and the feature points and the values thereof to the external device 30 in step S105. The control unit 10 determines, in step S106, whether or not an instruction to end measurement has been received from the external device 30, and repeats the processing from step S102 until the instruction to end measurement is received.

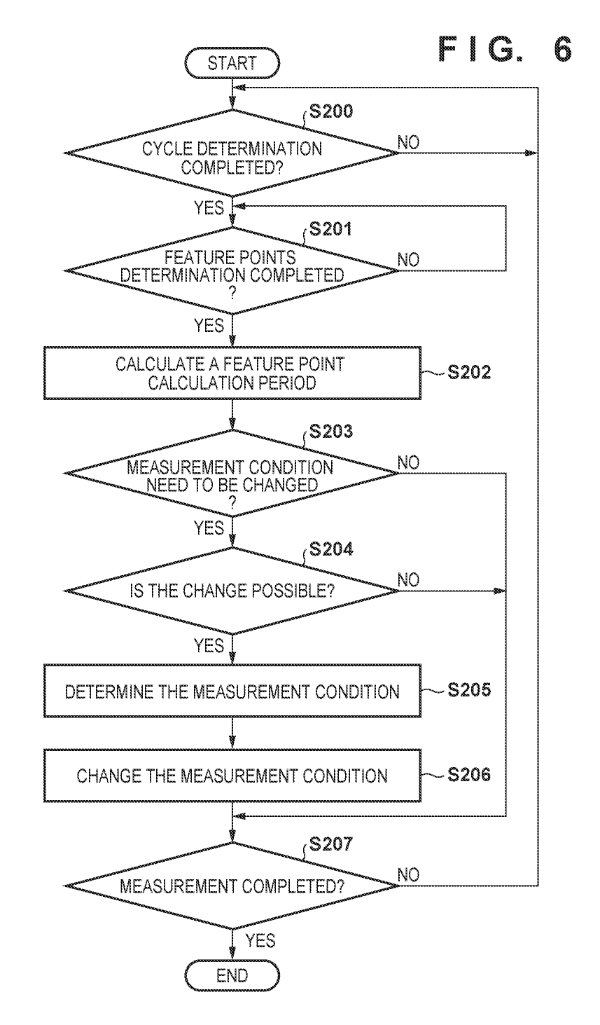

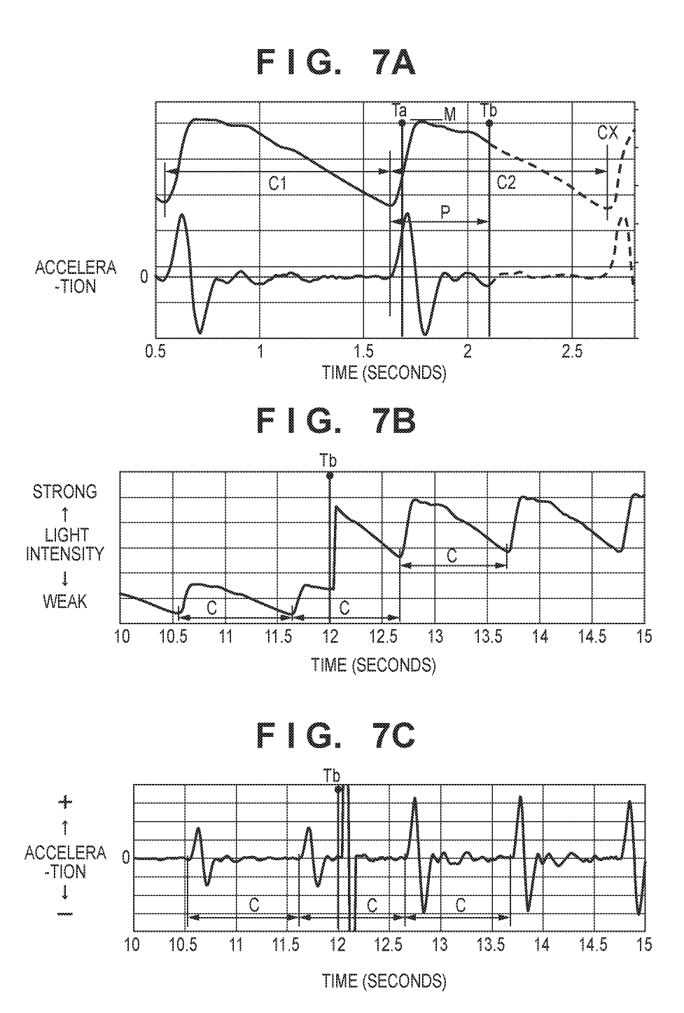

FIG. 6 is a flowchart of processing for changing the measurement condition that is performed while the processing from step S102 to S105 in FIG. 4 is repeated. In step S200, the condition change unit 14 waits until the cycle calculation unit 15 completes calculation of the cycle C of the biological signal. FIG. 7A shows a state in which the cycle calculation unit 15 has detected the end of the cycle C1 of the biological signal at a timing Ta. In the present embodiment, the cycle calculation unit 15 detects a local minimum of the biological signal by detecting continuous increase of the amplitude of the biological signal for a predetermined time period or more. That is, the cycle calculation unit 15 detects the ending time (time of local minimum) of the cycle C1 of the biological signal after this event.

Upon detecting the end of the previous cycle of the biological signal at the timing Ta, the condition change unit 14 waits until the feature calculation unit 16 completes calculation of feature points at the current cycle C (cycle C2 in FIG. 7A) in step S201. The timing at which calculation of feature points has ended is shown as timing Tb in FIG. 7A. Upon the calculation of feature points being completed, the condition change unit 14 calculates the period from the start timing of the current cycle C2 to the end of calculation of the feature points of the biological signal as a feature point calculation period P in step S202. The feature point calculation period P is a period from the end timing (start timing of the cycle C2) of the cycle C1 to the timing Tb. The condition change unit 14 determines, in step S203, whether or not the measurement condition needs to be changed. For example, the condition change unit 14 obtains a peak value M of the biological signal in the feature point calculation period P in the current cycle, and determines that the measurement condition needs to be changed if the peak value M is not in a predetermined range. For example, when the AD conversion circuit 55 has 12-bit resolution, the predetermined range may be 3800 to 4000, which is close to the maximum value that can be detected by the AD conversion circuit 55.

The condition change unit 14, upon determining that the measurement condition need not be changed, determines whether or not the measurement of the biological information has ended in step S207, and repeats the processing from step S200 if not ended. On the other hand, upon determining that the measurement condition needs to be changed, the condition change unit 14 determines whether or not the measurement condition can be changed in step S204. In the present embodiment, the condition change unit 14 estimates the ending time of the current cycle C2 as an estimated time CX, and determines that the measurement condition can be changed if the change of the measurement condition will be completed until the estimated time CX. On the other hand, if the change of the measurement condition will not be completed until the estimated time CX, the condition change unit 14 determines that measurement condition cannot be changed. In the present embodiment, the estimated time CX is a time after the start timing of the cycle C2 by the period corresponding to the previous cycle C1. Also, the configuration may be such that the estimated time CX is calculated using an average value of a plurality of past cycles C, instead of the previous cycle C. Note that the cycle of the biological signal varies, and therefore, the condition change unit 14 can use a variation margin Z, which is the variation amount of the biological signal, to calculate the estimated time CX. In this case, the condition change unit 14 determines the estimated time CX to be a time prior, by the variation margin Z, to the time that is after the start timing of the cycle C2 by the period of the previous cycle or the average value of the periods of a plurality of past cycles. Also, the value of the variation margin Z is pre-stored in the ROM 51. In this example, the variation margin Z is 100 milliseconds.

The condition change unit 14 obtains a total value S1 of the feature point calculation period P and a change time period D needed to change the measurement condition, and determines the time that is after the start timing of the cycle C2 by the total value S1 as a completion time CP. Then, the condition change unit 14 determines that the measurement condition cannot be changed if the completion time CP is after the estimated time CX, and if not, determines that the measurement condition can be changed. In other words, the condition change unit 14 determines whether or not the change of the measurement condition can be completed in a period from the timing Tb at which the calculation of the feature amounts has completed until the estimated time CX by comparing the period with the change time period D. That is, the condition change unit 14 determines that the measurement condition cannot be changed if the period is shorter than the change time period D, and if not, determines that the measurement condition can be changed. Here, the change time period D is a period from the timing at which the measurement condition has been changed until the biological signal stabilizes such that the cycle and feature points can be determined based on the biological signal. For example, if the light emission intensity of the white light source 21 is changed, the wait period until the light emission intensity stabilizes is included in the change time period D. Also, if the received light amount detection unit 12 includes a filter circuit such as a low pass filter, a period based on the time constant of the filter is included in the change time period D. Moreover, if the biological signal generation unit 13 generates the biological signal by performing moving average processing or the like on digital values in time series, a period needed to perform averaging processing is included in the change time period D. In the present embodiment, only the light emission intensity of the white light source 21 is changed during measurement, and the biological signal generation unit 13 generates the biological signal by performing moving average processing on digital values in time series. In this case, the wait period until the light emission intensity stabilizes is 20 milliseconds, the period needed to perform moving average processing is 80 milliseconds, and the change time period D can be set to 100 milliseconds, which is the sum thereof.

Note that, if the decrease of the amplitude of the biological signal before taking a local minimum value needs to be detected for a predetermined period in order to detect the local minimum of the biological signal, the condition change unit 14 can consider this predetermined period as a detection time period Y in which the total value S1 is calculated. In this case, the condition change unit 14 can determine the completion time CP by obtaining the total value S1 of the feature point calculation period P, the change time period D, and the detection time period Y. That is, the condition change unit 14 determines that the measurement condition cannot be changed if the period from the timing Tb at which the calculation of feature amounts has been completed until the estimated time CX is shorter than the sum of the change time period D and the detection time period Y, and if not, determines that the measurement condition can be changed.

Returning to FIG. 6, upon determining that the measurement condition can be changed in step S204, the condition change unit 14 calculates, in step S205, a new measurement condition based on the maximum value M of the biological signal in the feature point calculation period P. In this example, the light emission intensity of the white light source 21 is changed and the charge accumulation time period of the line sensor 24 is fixed during measurement. The method of determining the measurement condition is similar to that described in step S100 in FIG. 4, and repetitive description thereof is omitted. The condition change unit 14 changes the measurement condition to that determined in step S206. FIG. 7B shows an example of the biological signal when the measurement condition is changed at the timing Tb. FIG. 7C shows an example of the acceleration signal when the measurement condition is changed at the timing Tb. The condition change unit 14 repeats the processing from step S200 until the instruction to end measurement is received from the external device 30.

For example, the pressing force of a finger, which is the measurement target 90, to the transparent cover 400 of the aperture portion 500 and the contact condition between the transparent cover 400 and the measurement target 90 may change during measurement. In this case, the measurement condition determined when the measurement started may not be appropriate to the measurement target 90 after the state has changed. Therefore, in the present embodiment, the measurement condition is changed as necessary during the measurement. Here, in the present embodiment, it is determined whether or not the measurement condition can be changed in a period from a timing at which feature points have been calculated in a certain cycle of the biological signal until the start of the next cycle. If the measurement condition can be changed before the next cycle starts, the measurement condition is changed. That is, if the measurement condition can be changed in a signal period of the biological signal that is not used to detect the biological information, the measurement condition is changed. According to this configuration, even in a case where the state of the measurement target 90 has changed, the measurement condition can be changed without interrupting the calculation of the feature points and cycle of the biological signal.

Note that the case of “No” in step S204 in FIG. 6 corresponds to a case where although it has been determined that the measurement condition needs to be changed, the measurement condition cannot be changed, for example. Therefore, the configuration may also be such that, if the result of processing in step S204 in FIG. 6 is “No” successively a predetermined number of times, the measurement condition is mandatorily changed. In this case, although the calculation of feature points in one cycle is interrupted, the detection accuracy of the biological signal can be suppressed from degrading due to the fact that the measurement condition is not appropriate.

### Second Embodiment

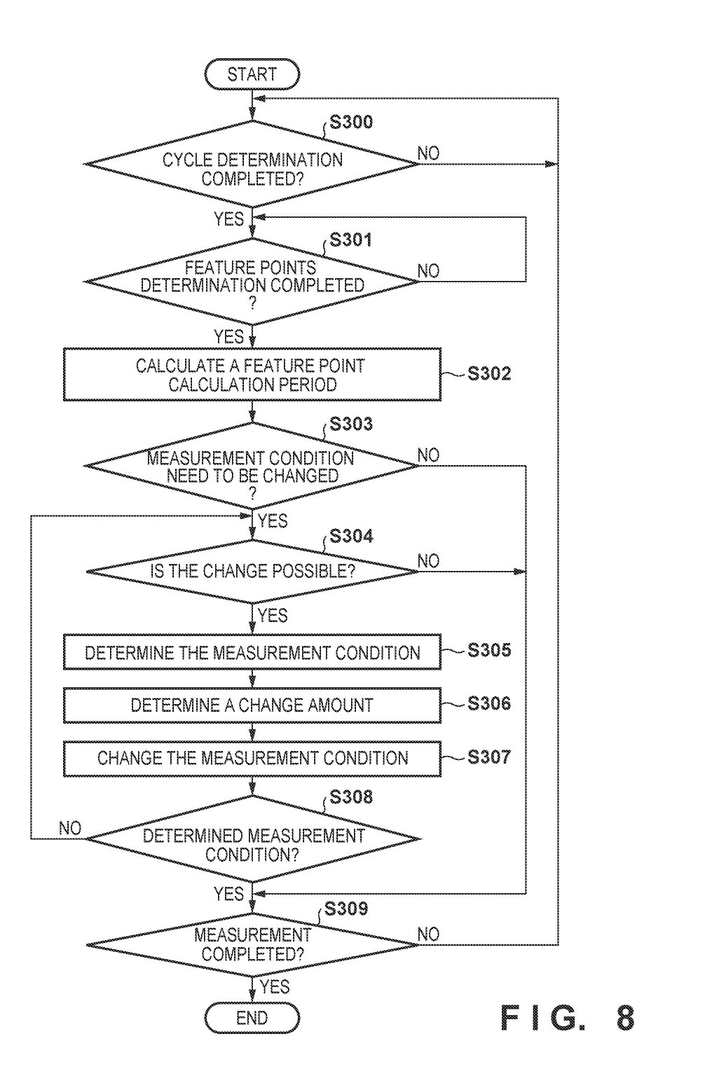

Next, a second embodiment will be described focusing on differences with the first embodiment. In the first embodiment, the revised measurement condition is determined in step S205 in FIG. 6, and the measurement condition is changed to the measurement condition determined in step S206. In the present embodiment, the change amount of the measurement condition is limited. FIG. 8 is a flowchart of processing for changing the measurement condition according to the present embodiment. The processing from step S300 to step S303 is the same as the processing from step S200 to step S203 in FIG. 6, and description thereof is omitted.

If it has been determined, in step S303, that the measurement condition need to be changed, a condition change unit 14 determines, in step S304, whether or not the change of the measurement condition will be completed before the current cycle ends. In the present embodiment as well, the condition change unit 14 obtains an estimated time CX, and determines the time after the current time by a change time period D as a completion time CP, similarly to the first embodiment. Then, the condition change unit 14 determines that the measurement condition cannot be changed if the completion time CP is after the estimated time CX, and if not, determines that the measurement condition can be changed. If it has been determined that the measurement condition can be changed in step S304, the condition change unit 14 determines a new measurement condition in step S305. The method of determining the new measurement condition is similar to that in the first embodiment. Next, the condition change unit 14 determines the change amount of the measurement condition in step S306. For example, if the light emission intensity of a white light source 21 is changed, the condition change unit 14 obtains the upper limit of the change amount by calculating a certain percentage of the intensity before change. The percentage can be 0.5%, for example. Also, the condition change unit 14 determines, if the amount of change of the measurement condition determined in step S305 exceeds this upper limit, that this upper limit is the change amount. On the other hand, if the amount of change of the measurement condition determined in step S305 does not exceed the upper limit, the amount of change to be determined in step S306 is the amount of change of the measurement condition determined in step S305. Note that the upper limit of a change amount can also be specified using an absolute value instead of a percentage. Note that the upper limit values of change amounts or parameters used for determining the upper limit values are pre-stored in a ROM 51.

The condition change unit 14 changes, in step S307, the measurement condition by the change amount determined in step S306. Then, the condition change unit 14 determines, in step S308, whether or not the revised measurement condition is the measurement condition determined in step S305. If the revised measurement condition is the measurement condition determined in step S305, the condition change unit 14 determines, in step S309, whether or not the measurement of biological information has ended. On the other hand, if the revised measurement condition has not reached the measurement condition determined in step S305, the condition change unit 14 repeats the processing from step S304. Note that, if the processing is repeated from step S304, the condition change unit 14 sets a timing after the timing at which the measurement condition has been changed in step S307 by a predetermined time period W to the current time, which is the reference point when the completion time CP is calculated, in the next determination in step S304. Note that the predetermined time period W can be the same as the change time period D. That is, if the measurement condition is changed a plurality of times in one cycle of the biological signal, the condition change unit 14 secures the predetermined time period W as the interval of timings at which the measurement condition is changed. The reason for this is to make it possible to perform the next change of the measurement condition after the biological signal has stabilized after the previous change of the measurement condition.

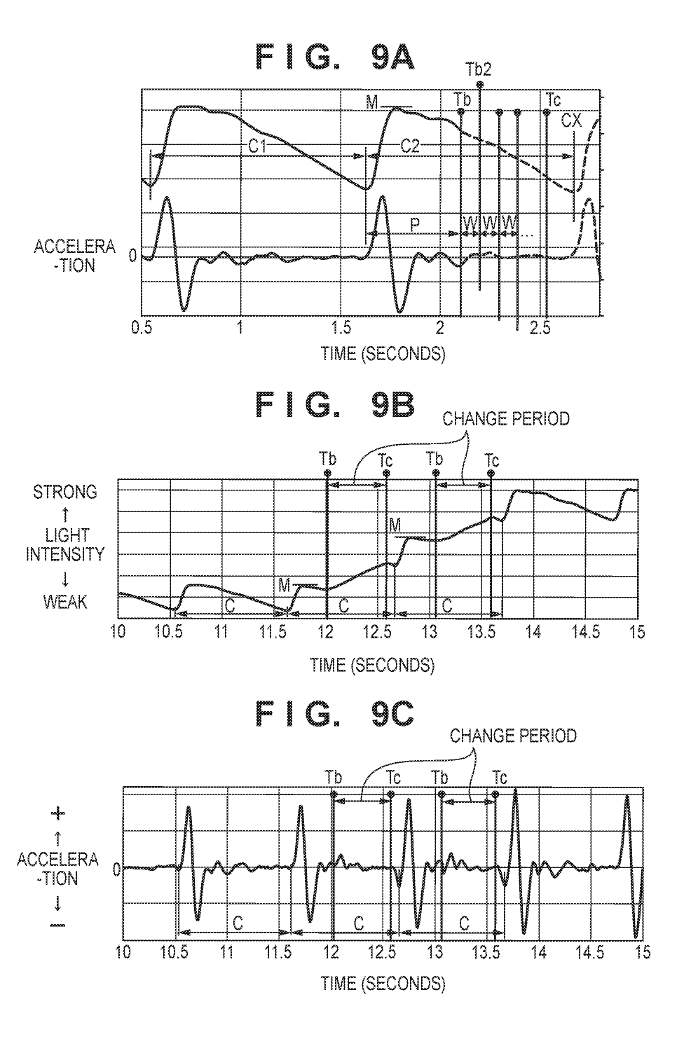

FIG. 9A shows a manner in which the measurement condition is changed a plurality of times in a period from a timing Tb at which feature points were calculated until a timing Tc. The intervals of temporally adjacent change timings of the measurement condition are the predetermined time period W, as described above. FIG. 9B shows an example of the biological signal when, in each of two cycles of the biological signal, the measurement condition is changed in a period from a timing Tb to a timing Tc. FIG. 9C shows an example of the acceleration signal when, in each of two cycles of the biological signal, the measurement condition is changed a plurality of times in a period from a timing Tb to a timing Tc.

As described above, in the present embodiment, as a result of providing an upper limit to the change amount of the measurement condition, the change in the biological signal and the acceleration signal with respect to the change of the measurement condition can be suppressed. For example, when the plethysmogram signal is detected as the biological signal, and the measurement is performed while the acceleration signal is displayed in the external device 30, unnecessary signal changes at positions other than the feature points of the acceleration signal can be reduced. Also, when the pulse rate is calculated from the intervals of local maximums of the acceleration signal, the possibility that the pulse rate is erroneously detected due to the change in the acceleration signal that is caused by the change of the measurement condition can be reduced.

### Third Embodiment

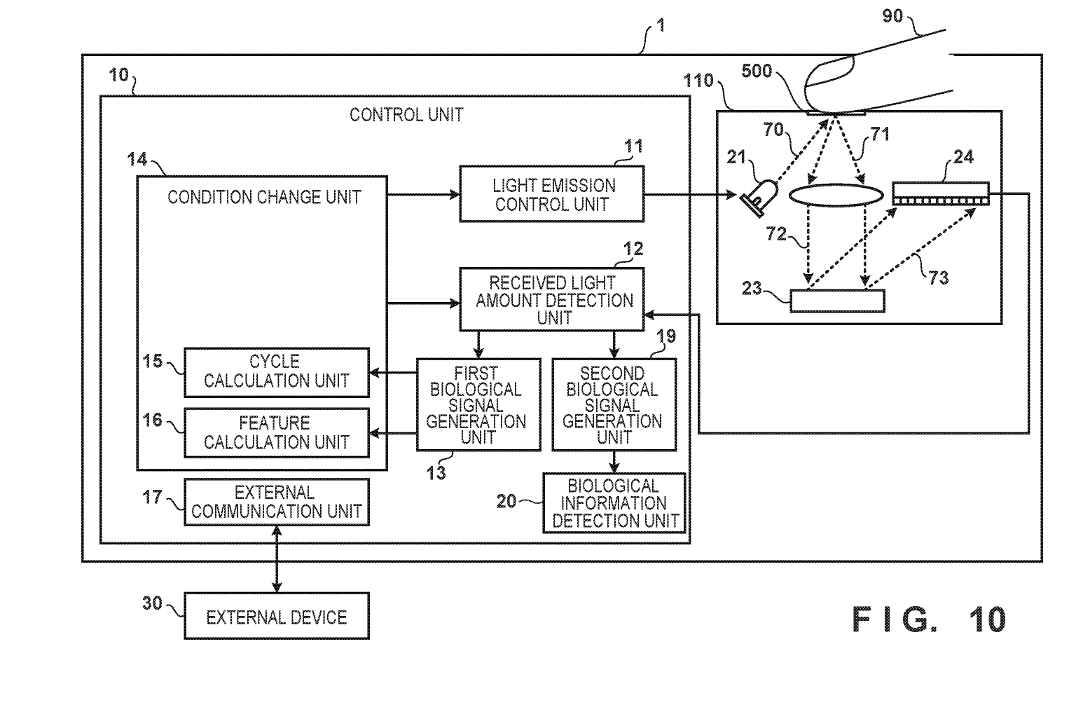

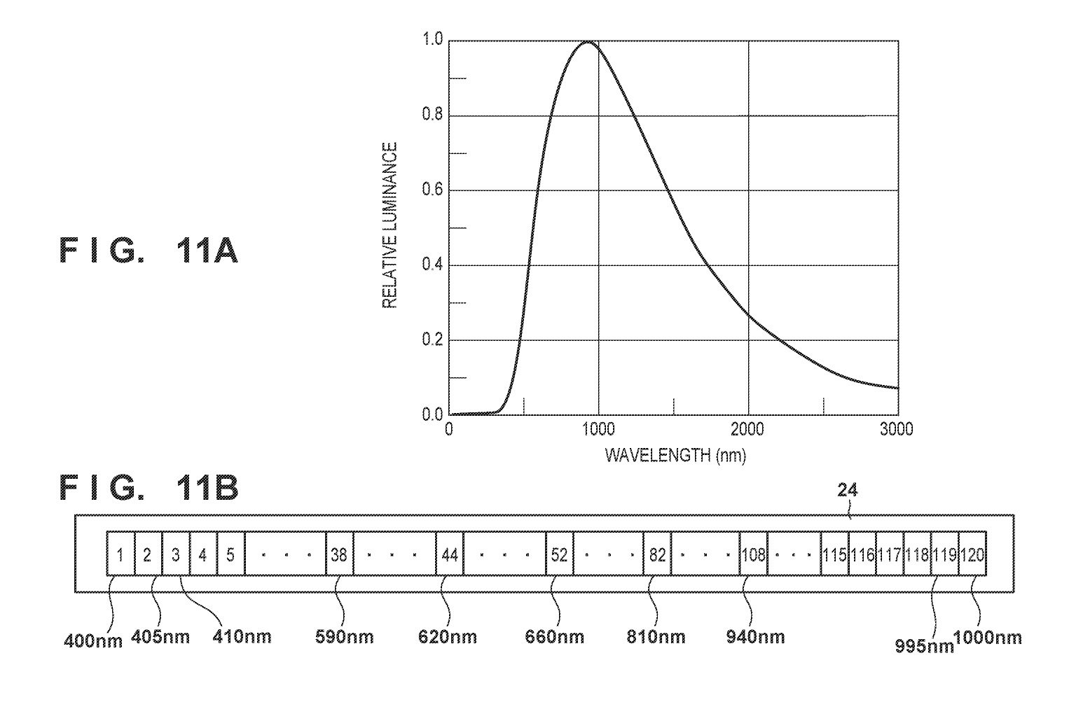

Next, a third embodiment will be described focusing on differences with the first embodiment. FIG. 10 is a block diagram for describing operations of a measurement apparatus 1 in the present embodiment. Note that constituent elements that are similar to those in the block diagram in FIG. 1 are given the same reference signs, and redundant descriptions will be omitted. In the present embodiment, a white light source 21 is a white LED using a tungsten light. FIG. 11A shows relative intensity (luminance) of the white light source 21 used in the present embodiment for each wavelength. FIG. 11B is a schematic diagram of a line sensor 24 in the present embodiment. The line sensor 24 according to the present embodiment includes 120 pixels needed to detect visible light in a wavelength range from about 400 nm to about 1000 nm in units of 5 nm. In the present embodiment, the measurement apparatus 1 is calibrated and assembled such that a first pixel of the line sensor 24 detects light including light having wavelength of about 400 nm and a 120^{th} pixel detects light including light having wavelength of about 1000 nm.

A first biological signal generation unit 13 is similar to the biological signal generation unit 13 in the first embodiment. However, because two biological signal generation units are present in the present embodiment, the biological signal generation unit 13 in the first embodiment is referred to as a first biological signal generation unit 13 in the present embodiment. The first biological signal generation unit 13 generates a first biological signal based on the received light amount of a 38^{th} pixel that detects light having a wavelength of about 590 nm, a fingertip of a living body being a measurement target 90.

A second biological signal generation unit 19 generates a second biological signal that indicates respective temporal changes of the received light amounts of a 52^{th} pixel that detects light having a wavelength of about 660 nm and a 108^{th} pixel that detects light having a wavelength of about 940 nm, of the plurality of pixels of the line sensor 24, and outputs the second biological signal to a biological information detection unit 20. The biological information detection unit 20 determines the percutaneous arterial blood oxygen saturation (SpO2) based on the second biological signal. The SpO2 indicates the ratio of hemoglobin molecules in arterial blood that are bound with oxygen molecules as a percentage. In the detection of the SpO2, the biological information detection unit 20 obtains the value R for the SpO2 from an equation R=P660/P940. Here, P660 is a received light amount of the 52^{th} pixel (about 660 nm), and P940 is a received light amount of the 108^{th} pixel (about 940 nm). Note that the SpO2 is obtained from the received light amount of the 52^{th} pixel and the received light amount of the 108^{th} pixel at the same timing. The biological information detection unit 20 determines the SpO2 by obtaining the value of the SpO2, in a calibration curve between the value R and the SpO2 that has been created in advance, at the value R obtained based on the second biological signal.

An external communication unit 17 corresponds to an external communication circuit 56, and communicates with an external device 30. The external device 30 instructs the measurement apparatus 1 to start and end measurement. Also, the measurement apparatus 1 transmits a first biological signal, a signal obtained by differentiating the first biological signal a plurality of times, a cycle C of the first biological signal, feature points of the first biological signal, the determined SpO2, and the like to the external device 30. Note that the external device 30 can calculate the pulse rate from the cycle C of the first biological signal. Moreover, the external device 30 can determine the degree of blood vessel stiffness based on the feature points of the first biological signal and the values thereof. Note that the configuration may be such that the second biological signal is transmitted to the external device 30, and the external device 30 determines the SpO2, instead of determining the SpO2 inside the control unit.

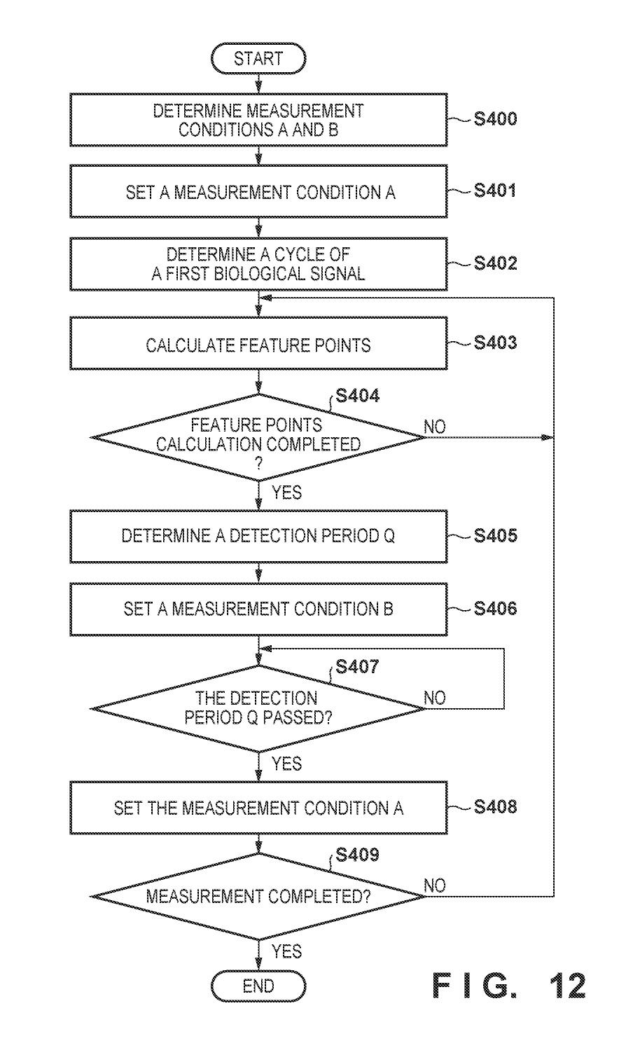

FIG. 12 is a flowchart of processing for detecting the biological information according to the present embodiment. The measurement apparatus 1, upon receiving an instruction to start measurement from the external device 30, determines measurement conditions A and B in step S400. Note that the measurement conditions are a light emission intensity of the white light source 21 and a charge accumulation time period (light receiving time period) of the line sensor 24, for example. Also, the measurement condition A is a measurement condition for generating the first biological signal, and the measurement condition B is a measurement condition for generating the second biological signal.

First, determination of the measurement condition A will be described. For example, a light emission control unit 11 causes the white light source 21 to emit light at a predetermined light emission intensity, and a received light amount detection unit 12 detects the received light amount of the 38^{th} pixel that is used to generate the first biological signal during a predetermined period. Also, the control unit 10 determines the light emission intensity of the white light source 21 and/or the charge accumulation time period of the line sensor 24 such that the maximum value (peak value) of the received light amount detected during the predetermined period is close to the maximum value of the voltage range that can be detected by the AD conversion circuit 55, and sets these values as the measurement condition A. Note that the predetermined period is set to be one cycle or more of a fingertip plethysmogram signal, which is the first biological signal to be generated, and may be two seconds, for example. Note that, the light emission intensity of the white light source 21 that is used in order to determine the measurement condition A in step S400 is determined in advance. Also, the control unit 10 determines the measurement condition A based on this light emission intensity and the maximum value of the received light amount of the 38^{th} pixel.

Next, determination of the measurement condition B will be described. The second biological signal is generated based on the received light amount of the 52^{th} pixel (about 660 nm) and the received light amount of the 108^{th} pixel (about 940 nm), as described above. Therefore, similarly to the determination of the measurement condition A, the light emission control unit 11 causes the white light source 21 to emit light at a predetermined light emission intensity, and the received light amount detection unit 12 detects the received light amounts of the 52^{th} and 108^{th} pixels during a predetermined period. Also, the control unit 10 determines the light emission intensity of the white light source 21 and/or the charge accumulation time period of the line sensor 24 such that the received light amounts of the 52^{th} and 108^{th} pixels detected during the predetermined period are close to the maximum value of the voltage range that can be detected by the AD conversion circuit 55, and sets these values as the measurement condition B. Note that the measurement conditions A and B can be determined in parallel, or separately. Also, the configuration may be such that the measurement condition is determined such that the maximum value of the received light amount of a pixel to be used to generate the biological signal is appropriate while changing the light emission intensity of the white light source 21 and/or the charge accumulation time period of the line sensor 24 for each predetermined period.

The control unit 10 sets the determined measurement condition A to the light emission control unit 11 and the received light amount detection unit 12 in step S401. With this, the light emission control unit 11 causes the white light source 21 to emit light at a light emission intensity according to the measurement condition A. Also, the received light amount detection unit 12 sets the charge accumulation time period according to the measurement condition A to the line sensor 24. The first biological signal generation unit 13 generates the first biological signal based on the received light amounts of the 38^{th} pixel at respective timings, and outputs the first biological signal to a cycle calculation unit 15 and a feature calculation unit 16.

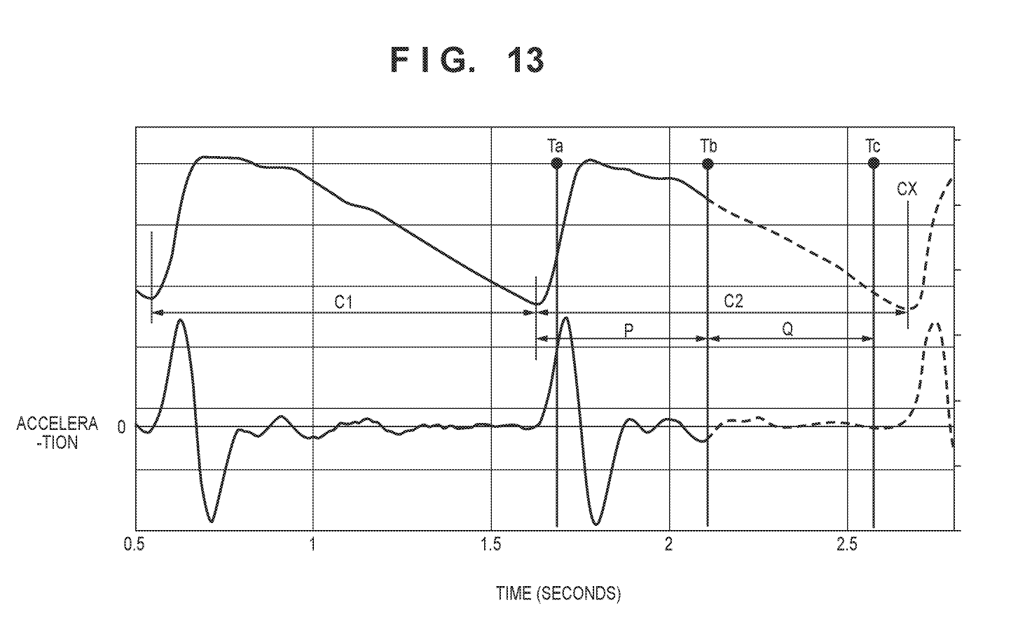

The processing performed in the cycle calculation unit 15 and the feature calculation unit 16 is similar to that in the first embodiment. When the feature calculation unit 16 has completed calculation of all feature points, a condition change unit 14 determines, in step S405, a detection period Q of the second biological signal. FIG. 13 is a diagram illustrating the detection period Q. The timing Ta in FIG. 13 is a timing at which the cycle calculation unit 15 has detected the end of a cycle C1 of the first biological signal, that is, a local minimum. The timing Tb in FIG. 13 is a timing at which the feature calculation unit 16 has completed calculation of all the feature points. Also, the time CX is an estimated time at which the current cycle C2 is estimated to end. The condition change unit 14 estimates the time that is after the start timing of the cycle C2 by the period corresponding to the previous cycle C1 as an estimated time CX. Also, the configuration may be such that the estimated time CX is calculated using an average value of a plurality of past cycles C, instead of the previous cycle C. Note that the cycle of the first biological signal varies, and therefore, the condition change unit 14 can use a variation margin Z, which is the variation amount of the first biological signal, to calculate the estimated time CX. In this case, the condition change unit 14 determines the estimated time CX to be a time prior, by the variation margin Z, to the time that is after the start timing of the cycle C2 by the length of the previous cycle or the average value of the lengths of a plurality of past cycles. Note that the value of the variation margin Z is pre-stored in a ROM 51.

The condition change unit 14 obtains a timing Tc that is before the estimated time CX by a change time period D, and determines that the timing Tc is the end timing of the detection period. Also, the condition change unit 14 determines the period from the timing Tb to the timing Tc as a detection period Q. The change time period D is similar to that in the first embodiment. Note that, if the decrease in the amplitude of the biological signal before taking a local minimum value needs to be detected for a predetermined period in order to detect the local minimum of the biological signal, the condition change unit 14 can use this predetermined period as a detection time period Y to calculate the timing Tc. In this case, the condition change unit 14 determines a timing before the estimated time CX by the sum of the change time period D and the detection time period Y as the timing Tc.

Returning to FIG. 12, the control unit 10 sets the measurement condition B that has been determined in step S406 (timing Tb) to the light emission control unit 11 and the received light amount detection unit 12. When a change period from the measurement condition A to the measurement condition B has elapsed after the measurement condition was switched to the measurement condition B, the second biological signal generation unit 19 generates the second biological signal, and outputs the second biological signal to the biological information detection unit 20. With this, the biological information detection unit 20 determines the SpO2 based on the received light amounts of the 52^{th} pixel (about 660 nm) and the 108^{th} pixel (about 940 nm) that are indicated by the second biological signal. Note that, because the line sensor 24 outputs a received light amount for each charge accumulation time period, the biological information detection unit 20 determines the SpO2 a plurality of times during the detection period Q. Also, when the detection period Q has elapsed, the biological information detection unit 20 obtains an average value of the plurality of pieces of SpO2 data obtained during the detection period.

The control unit 10, after setting the measurement condition B in step S406, waits until the detection period Q has elapsed, in step S407. When the detection period Q has elapsed, the control unit 10 sets the measurement condition A to the light emission control unit 11 and the received light amount detection unit 12 in step S408 (timing Tc). Also, the control unit 10 determines, in step S409, whether or not an instruction to end measurement has been received from the external device 30, and repeats the processing from step S403 until the instruction to end measurement has been received. Note that, every time one cycle of the first biological signal has completed, the external communication unit 17 outputs the acceleration signal, the cycle C of the biological signal, the feature points and the values thereof, and the SpO2 average value to the external device 30.

As described above, a measurement condition appropriate for generating the first biological signal is set in a period during which the biological information is measured by generating the first biological signal. Also, the estimated time CX at which the current cycle of the first biological signal is estimated to end is obtained based on the past one or more cycles of the first biological signal. Also, when feature points have been calculated from the first biological signal of the current cycle, the detection period Q of the biological information based on the second biological signal is determined based on the timing at which the feature points have been calculated and the estimated time CX. Note that, here, the period needed to change the measurement condition is taken into consideration. Also, a measurement condition appropriate for generating the second biological signal is set in the detection period Q. According to this configuration, the SpO2 can be accurately determined based on the second biological signal while continuing the calculation of feature points from the first biological signal. That is, without interrupting the measurement, measurement conditions appropriate for a plurality of pieces of biological information of a detection target can be used, and the detection accuracy of each biological information can be improved. Note that, in the present embodiment, the detection period Q includes a change period needed to change the measurement condition from the measurement condition A to the measurement condition B. However, the configuration may be such that the change period from the measurement condition A to the measurement condition B is not included in the detection period Q. In this case, the start timing of the detection period Q is a timing after the timing Tb by the change period from the measurement condition A to the measurement condition B.

### Fourth Embodiment

Next, a fourth embodiment will be described focusing on differences with the third embodiment. In the present embodiment, a biological information detection unit 20 determines carboxyhemoglobin concentration (SpCO) in addition to SpO2. The SpCO can be detected based on received light amounts of a 44^{th} pixel that detects light having a wavelength of about 620 nm, a 52^{th} pixel that detects light having a wavelength of about 660 nm, an 82^{th} pixel that detects light having a wavelength of about 810 nm, and a 108^{th} pixel that detects light having a wavelength of about 940 nm. Note that the method of determining the SpCO based on the received light amounts at four wavelengths is described in Japanese Patent Laid-Open No. 2015-000127, for example.

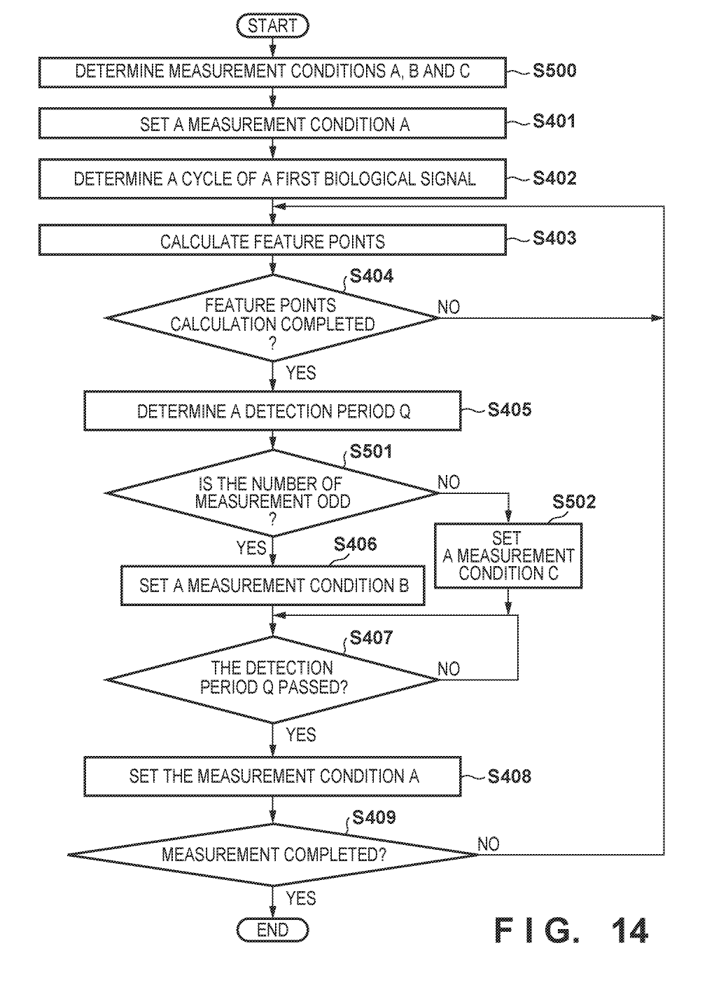

FIG. 14 is a flowchart of processing for detecting biological information according to the present embodiment. Note that steps similar to those in the flowchart in FIG. 12 are given the same step numbers, and the description thereof will be omitted. A measurement apparatus 1, upon receiving an instruction to start measurement from an external device 30, determines measurement conditions A, B, and C, in step S500. The measurement conditions A and B are similar to those in the third embodiment. The measurement condition C is a condition for measuring the SpCO, and is a condition for making the maximum value of received light amounts of the four pixels that respectively receive light fluxes of different wavelengths for detecting the SpCO to be close to the maximum value of the voltage range that can be detected by an AD conversion circuit 55.

Upon determining a detection period Q in step S405, a condition change unit 14 determines, in step S501, whether or not the measurement is an odd-number-th measurement. If the measurement is an odd-number-th measurement, the condition change unit 14 sets the measurement condition B to a light emission control unit 11 and a received light amount detection unit 12 in step S406. Also, a second biological signal generation unit 19 generates a second biological signal that indicates received light amounts of the 52^{th} pixel (about 660 nm) and the 108^{th} pixel (about 940 nm). With this, the biological information detection unit 20 determines the SpO2 during the detection period Q. On the other hand, if the measurement is an even-number-th measurement, the condition change unit 14 sets the measurement condition C to the light emission control unit 11 and the received light amount detection unit 12 in step S502. Also, the second biological signal generation unit 19 generates a second biological signal that indicates received light amounts of the 44^{th} pixel (about 620 nm), the 52^{th} pixel (about 660 nm), the 82^{th} pixel (about 810 nm), and the 108^{th} pixel (about 940 nm). With this, the biological information detection unit 20 determines the SpCO during the detection period Q. Note that, in the present embodiment, an external communication unit 17 alternatingly output the SpO2 and the SpCO, for each cycle of a first biological signal, to the external device 30.

As described above, in the present embodiment, pixels for generating the second biological signal are switched for each cycle of the first biological signal. According to this configuration, the number of types of biological information to be detected can be increased without the detection accuracy being degraded. Note that the number of pieces of biological information to be detected using the second biological signal can be three or more. In this case also, measurement conditions for respective pieces of biological information are obtained, and one measurement condition is sequentially selected and set for each cycle of the first biological signal. Note that information indicating the measurement sequence of the plurality of types of biological information are pre-stored in a ROM 51.

Note that above-described embodiments have been described assuming that the measurement condition includes the light emission intensity of the white light source 21 and the charge accumulation time period of the line sensor 24, as an example. The measurement condition is not limited thereto, and the configuration may be such that the measurement condition includes the light reception sensitivity (gain) of the line sensor 24, for example. Also, in the fourth embodiment, only the SpO2 or the SpCO is detected in one detection period Q. However, the configuration may be such that one detection period Q is divided into a first half and a second half, according to the time of the detection period Q, and the SpO2 is detected in the first half, and the SpCO is detected in the second half.

### Other Embodiments

Note that, in the above-described embodiments, the line sensor 24 of the measurement apparatus 1 receives reflected light from a measurement target 90, but the configuration may be such that the line sensor 24 receives transmitted light. Also, the external appearance and mechanical structure of the measurement apparatus 1 of the present invention are not limited to those shown in FIGS. 15A and 15B.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-060732, filed on Mar. 27, 2018, and Japanese Patent Application No. 2018-060735, filed on Mar. 27, 2018 which are hereby incorporated by reference herein in their entirety.

What is claimed is:

1. A measurement apparatus comprising:
a spectral sensor that includes a light source configured to emit light toward a measurement position, a spectral unit configured to disperse reflected light from a living body at the measurement position or transmitted light that has passed through a living body at the measurement position according to a wavelength, and a light receiving unit including a plurality of pixels, wherein each pixel of the plurality of pixels is configured to receive light having a predetermined wavelength that has been dispersed by the spectral unit;
a generation unit configured to generate a biological signal from a light reception result of a predetermined pixel of the light receiving unit;
a first determination unit configured to determine a cycle of the biological signal;
a detection unit configured to detect a feature amount of the biological signal;
a setting unit configured to set a measurement condition of the living body to the spectral sensor;
a second determination unit configured to determine whether or not the measurement condition needs to be changed based on the biological signal; and
a third determination unit configured to determine, if the second determination unit has determined that the measurement condition needs to be changed, in a first cycle of the biological signal, whether or not the measurement condition can be changed in a period from a timing at which the detection unit has detected the feature amount in the first cycle until an ending time of the first cycle.

2. The measurement apparatus according to claim 1, wherein the third determination unit is further configured to estimate the ending time of the first cycle based on one or more second cycles before the first cycle.

3. The measurement apparatus according to claim 2, wherein the third determination unit is further configured to estimate the ending time of the first cycle additionally based on a variation amount of the cycle of the biological signal.

4. The measurement apparatus according to claim 1, wherein the third determination unit is further configured to determine whether or not the measurement condition can be changed by comparing a time period needed to change the measurement condition with a period from a timing at which the detection unit has detected the feature amount in the first cycle until the ending time of the first cycle.

5. The measurement apparatus according to claim 1,
wherein the first determination unit is further configured to determine the cycle of the biological signal based on an extreme value of the biological signal, and
the third determination unit is further configured to determine whether or not the measurement condition can be changed by obtaining a sum of a time period needed to change the measurement condition and a predetermined time period needed for the first determination unit to detect an extreme value of the biological signal, and comparing the sum with a period from a timing at which the detection unit has detected the feature amount in the first cycle until the ending time of the first cycle.

6. The measurement apparatus according to claim 1, wherein the second determination unit is further configured to determine that, if a peak value in a cycle of the biological signal is not in a predetermined range, the measurement condition needs to be changed in the cycle.

7. The measurement apparatus according to claim 6,
wherein, if the second determination unit has determined that the measurement condition needs to be changed based on a peak value in the first cycle, the setting unit determines a revised measurement condition based on the peak value, and
the setting unit is further configured to, if the third determination unit has determined that the measurement condition can be changed in the first cycle, change the measurement condition to the revised measurement condition.

8. The measurement apparatus according to claim 6,
wherein, if the second determination unit has determined that the measurement condition needs to be changed based on a peak value in the first cycle, the setting unit determines a revised measurement condition based on the peak value,
the setting unit has a parameter for obtaining an upper limit value of a change amount of the measurement condition, and
the setting unit is further configured to, if the third determination unit has determined that the measurement condition can be changed in the first cycle, determine whether or not a change amount to the revised measurement condition exceeds the upper limit value, and if the change amount to the revised measurement condition exceeds the upper limit value, change the measurement condition by the upper limit value.

9. The measurement apparatus according to claim 8, wherein the third determination unit is further configured to determine, if the setting unit has changed the measurement condition by the upper limit value, whether or not the measurement condition can further be changed in the first cycle.

10. The measurement apparatus according to claim 1, wherein the measurement condition includes at least one of light emission intensity of the light source, a light receiving time period of the light receiving unit, and light reception sensitivity of the light receiving unit.

11. The measurement apparatus according to claim 1,
wherein the biological signal is a pulse wave signal, and
the detection unit detects a change point in a signal obtained by differentiating the pulse wave signal one or more times as the feature amount.

12. The measurement apparatus according to claim 11, wherein the detection unit obtains a differential signal by differentiating the pulse wave signal twice or four times, and detects a predetermined number of extreme values of the differential signal from a timing of an extreme value of the pulse wave signal as the feature amount.

13. A measurement apparatus comprising:
a spectral sensor that includes a light source configured to emit light toward a measurement position, a spectral unit configured to disperse reflected light from a living body at the measurement position or transmitted light that has passed through a living body at the measurement position according to a wavelength, and a light receiving unit including a plurality of pixels, wherein each pixel of the plurality of pixels is configured to receive light having a predetermined wavelength that has been dispersed by the spectral unit;
a generation unit configured to generate a biological signal from a light reception result of a predetermined pixel of the light receiving unit;
a detection unit configured to detect a feature amount of the biological signal; and
a setting unit configured to set a measurement condition of the living body to the spectral sensor,
wherein the setting unit is further configured to set, in a period from a timing at which the detection unit has detected the feature amount in a cycle of the biological signal until an ending time of the cycle, a measurement condition of the living body.

14. A non-transitory computer-readable storage medium storing a computer program, the computer program, upon being executed in one or more processors of a measurement apparatus that includes:
a spectral sensor that includes a light source configured to emit light toward a measurement position, a spectral unit configured to disperse reflected light from a living body at the measurement position or transmitted light that has passed through a living body at the measurement position according to a wavelength, and a light receiving unit including a plurality of pixels, wherein each pixel of the plurality of pixels is configured to receive light having a predetermined wavelength that has been dispersed by the spectral unit; and
the one or more processors,
causing the measurement apparatus to perform:
setting a measurement condition of the living body to the spectral sensor;
generating a biological signal from a light reception result of a predetermined pixel of the light receiving unit;
determining a cycle of the biological signal;
detecting a feature amount of the biological signal; and
determining whether or not the measurement condition needs to be changed, if having determined that the measurement condition needs to be changed in a first cycle of the biological signal, determining whether or not the measurement condition can be changed in a period from a timing at which the feature amount has been detected in the first cycle until an ending time of the first cycle.

15. A measurement apparatus comprising:
a spectral sensor that includes a light source configured to emit light toward a measurement position, a spectral unit configured to disperse reflected light from a living body at the measurement position or transmitted light that has passed through a living body at the measurement position according to a wavelength, and a light receiving unit including a plurality of pixels, wherein each pixel of the plurality of pixels is configured to receive light having a predetermined wavelength that has been dispersed by the spectral unit;
a generation unit configured to generate a biological signal from a light reception result of a first pixel of the light receiving unit;
a determination unit configured to determine a cycle of the biological signal;
a first detection unit configured to detect a feature amount of the biological signal in each cycle of the biological signal;
a second detection unit configured to detect biological information from a light reception result of a second pixel of the light receiving unit;
a determination unit configured to determine a first measurement condition for generating the biological signal and a second measurement condition to detect the biological information by the second detection unit; and
a setting unit configured to, in each cycle of the biological signal, determine an end timing of detection performed by the second detection unit, set the first measurement condition to the spectral sensor until the first detection unit has detected the feature amount, and set the second measurement condition to the spectral sensor in a period from when the first detection unit has detected the feature amount until the end timing.

16. The measurement apparatus according to claim 15, wherein the setting unit is further configured to estimate the ending time of a cycle of the biological signal based on one or more cycles before the cycle, and determine the end timing based on the estimated ending time.

17. The measurement apparatus according to claim 16, wherein the setting unit is further configured to estimate the ending time additionally based on a variation amount of the cycle of the biological signal.

18. The measurement apparatus according to claim 16, wherein the setting unit is further configured to determine a timing before the estimated ending time by a time period needed to change the measurement condition from the second measurement condition to the first measurement condition as the end timing.

19. The measurement apparatus according to claim 16,
wherein the determination unit is further configured to determine the cycle of the biological signal based on an extreme value of the biological signal, and
the setting unit is further configured to determine, as the end timing, a timing before the estimated ending time by a time period that is obtained by adding a time period needed to change the measurement condition from the second measurement condition to the first measurement condition and a predetermined time period needed for the determination unit to detect an extreme value of the biological signal.

20. The measurement apparatus according to claim 15,
wherein the setting unit is further configured to change, when the detection unit has detected the feature amount, the measurement condition to the second measurement condition, and
the second detection unit is further configured to detect, upon the measurement condition being changed to the second measurement condition, during a period after a timing at which a time period needed to change the measurement condition from the first measurement condition to the second measurement condition has elapsed until the end timing, the biological information based on the light reception result of the second pixel.

21. The measurement apparatus according to claim 20,
wherein a plurality of pixels of the light receiving unit are each the second pixel,
the second detection unit is further configured to detect a plurality of types of the biological information from the light reception results of the plurality of second pixels,
the determination unit determines a plurality of the second measurement conditions for detecting the respective plurality of types of biological information, and
the second detection unit is further configured to detect each of the plurality of types of biological information from the light reception result of at least one second pixel of the plurality of second pixels.

22. The measurement apparatus according to claim 21, wherein the determination unit is further configured to determine each second measurement condition for detecting the corresponding biological information based on a peak value of the received light amount of the corresponding at least one second pixel to be used to detect the biological information.

23. The measurement apparatus according to claim 21, further comprising a holding unit configured to hold information indicating a measurement sequence of the plurality of types of biological information,
wherein the setting unit is further configured to, in each cycle of the biological signal, select one biological information from the plurality of types of biological information according to the measurement sequence, and when the detection unit has detected the feature amount, change the measurement condition to the second measurement condition for detecting the selected biological information.

24. The measurement apparatus according to claim 15, wherein the determination unit is further configured to determine the first measurement condition based on the peak value of a received light amount of the first pixel.

25. The measurement apparatus according to claim 15, wherein the first measurement condition and the second measurement condition each include at least one of light emission intensity of the light source, a light receiving time period of the light receiving unit, and light reception sensitivity of the light receiving unit.

26. A non-transitory computer-readable storage medium storing a computer program, the computer program, upon being executed in one or more processors of a measurement apparatus that includes:
a spectral sensor that includes a light source configured to emit light toward a measurement position, a spectral unit configured to disperse reflected light from a living body at the measurement position or transmitted light that has passed through a living body at the measurement position according to a wavelength, and a light receiving unit including a plurality of pixels, wherein each pixel of the plurality of pixels is configured to receive light having a predetermined wavelength that has been dispersed by the spectral unit; and
the one or more processors,
causing the measurement apparatus to perform:
setting a first measurement condition to the spectral sensor;
generating a biological signal from a light reception result of a first pixel of the light receiving unit, in a state in which the first measurement condition has been set;
determining a cycle of the biological signal;
detecting a feature amount of the biological signal in each cycle of the biological signal;
determining an end timing of detection of biological information in each cycle of the biological signal;
setting, upon detecting a feature amount of the biological signal in each cycle of the biological signal, a second measurement condition to the spectral sensor; and
detecting, upon the second measurement condition being set to the spectral sensor, the biological information from a light reception result of a second pixel of the light receiving unit until the end timing.
